(19) United States
(12) Patent Application Publication
(10) Pub. No.: US 2025/0009720 A1
(43) Pub. Date: Jan. 9, 2025

(54) METHODS AND COMPOSITIONS FOR TREATING SLEEP APNEA

(71) Applicant: APNIMED, INC. (DELAWARE), Cambridge, MA (US)

(72) Inventors: Lawrence G. MILLER, Cambridge, MA (US); Ronald FARKAS, Clarksville, MD (US); Luigi TARANTO-MONTEMURRO, Brookline, MA (US); David P. WHITE, Denver, CO (US)

(21) Appl. No.: 18/708,616

(22) PCT Filed: Nov. 10, 2022
(86) PCT No.: PCT/US2022/049478

§ 371 Date: May 9, 2024

Related U.S. Application Data
(60) Provisional application No. 63/278,143, filed on Nov. 11, 2021.

(51) Int. Cl. A61K 31/433; A61K 31/138; A61K 31/496; A61K 31/5375

(52) U.S. Cl. CPC A61K 31/433; A61K 31/138; A61K 31/496; A61K 31/5375

(57) ABSTRACT

Pharmaceutical compositions comprising (i) a norepinephrine reuptake inhibitor (NRI) and (ii) a carbonic anhydrase inhibitor (CAI), in the absence of an antimuscarinic agent, and methods of treating conditions associated with pharyngeal airway collapse are described herein.

### CROSS-REFERENCE TO RELATED APPLICATION

The present application claims priority to and benefit of U.S. provisional application No. 63/278,143, filed Nov. 11, 2021, the entire contents of which are incorporated herein by reference.

### TECHNICAL FIELD

The present invention provides pharmaceutical compositions comprising (i) a norepinephrine reuptake inhibitor (NRI) or a hypnotic and (ii) a carbonic anhydrase inhibitor, and methods of treating sleep apnea.

### BACKGROUND

Obstructive Sleep Apnea (OSA) is a common disorder caused by collapse of the pharyngeal airway during sleep. OSA can have serious health consequences.

### SUMMARY

One aspect of the present invention provides a method of treating a subject having a condition associated with pharyngeal airway collapse, the method comprising administering to a subject in need thereof an effective amount of (i) a norepinephrine reuptake inhibitor (NRI) and (ii) a carbonic anhydrase inhibitor (CAI), in the absence of an antimuscarinic therapy.

Embodiments of this aspect of the invention may include one or more of the following optional features. In some embodiments, the NRI is a norepinephrine selective reuptake inhibitor (NSRI). In some embodiments, the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof. In some embodiments, the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof. In some embodiments, the NRI is reboxetine or a pharmaceutically acceptable salt thereof. In some embodiments, the NRI is atomoxetine or a pharmaceutically acceptable salt thereof. In some embodiments, the CAI is selected from the group consisting of acetazolamide, dichlorophenamide, dorzolamide, brinzolamide, methazolamide, zonisamide, ethoxzolamide, topiramate, sultiame, and any combination thereof, including pharmaceutically acceptable salts thereof. In some embodiments, the CAI is acetazolamide or a pharmaceutically acceptable salt thereof. In some embodiments, the NRI, such as atomoxetine or a pharmaceutically acceptable salt thereof, is administered at a dose of from about 20 to about 200 mg. In some embodiments, the NRI, such as atomoxetine or a pharmaceutically acceptable salt thereof, is administered at a dose of from about 25 to about 100 mg. In some embodiments, the CAI, such as acetazolamide, is administered at a dosage of from about 150 mg to about 750 mg. In some embodiments, the carbonic anhydrase inhibitor, such as acetazolamide, is administered at a dosage of about 500 mg. In some embodiments, the NRI and CAI are the only active agents administered in the method. In some embodiments, the NRI and CAI are administered in a single composition. In some embodiments, the single composition is an oral administration form. In some embodiments, the oral administration form is a syrup, pill, tablet, troche, capsule, or patch. In some embodiments, the condition associated the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state. In some embodiments, the non-fully conscious state is sleep.

Another aspect of the present invention provides a method of treating a subject having a condition associated with pharyngeal airway collapse, the method comprising administering to a subject in need thereof an effective amount of (i) a hypnotic and (ii) a carbonic anhydrase inhibitor (CAI), in the absence of an antimuscarinic therapy.

Embodiments of this aspect of the invention may include one or more of the following optional features. In some embodiments, the hypnotic is selected from the group consisting of temazepam, brotizolam, flurazepam, nitrazepam, and triazolam, and pharmaceutically acceptable salts thereof. In some embodiments, the hypnotic is selected from the group consisting of zolpidem, zopiclone, eszopiclone, gabapentin, trazodone, diphenhydramine, suvorexant, tasimelteon, ramelteon, agomelatine, doxepin, zaleplon, doxylamine, sodium oxybate, and tiagabine, and pharmaceutically acceptable salts thereof. In some embodiments, the hypnotic is trazodone or a pharmaceutically acceptable salt thereof. In some embodiments, the CAI is selected from the group consisting of acetazolamide, dichlorophenamide, dorzolamide, brinzolamide, methazolamide, zonisamide, ethoxzolamide, topiramate, sultiame, and any combination thereof, including pharmaceutically acceptable salts thereof. In some embodiments, the CAI is acetazolamide or a pharmaceutically acceptable salt thereof. In some embodiments, In some embodiments, the trazodone is administered at a dose of from about 12.5 to about 200 mg (or a dose equivalent of another hypnotic). In some embodiments, the trazodone is administered at a dose of from about 12.5 to about 50 mg (or a dose equivalent of another hypnotic). In some embodiments, the trazodone is administered at a dose of from about 50 to about 200 mg (or a dose equivalent of another hypnotic). In some embodiments, the trazodone is administered at a dose of from about 25 to about 100 mg (or a dose equivalent of another hypnotic). In some embodiments, the CAI, such as acetazolamide, is administered at a dosage of from about 150 mg to about 750 mg. In some embodiments, the carbonic anhydrase inhibitor, such as acetazolamide, is administered at a dosage of about 500 mg. In some embodiments, the hypnotic and CAI are the only active agents administered in the method. In some embodiments, the hypnotic and CAI are administered in a single composition. In some embodiments, the single composition is an oral administration form. In some embodiments, the oral administration form is a syrup, pill, tablet, troche, capsule, or patch. In some embodiments, the condition associated the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state. In some embodiments, the non-fully conscious state is sleep.

Another aspect of the present invention provides a pharmaceutical composition comprising (i) a norepinephrine reuptake inhibitor (NRI) and (ii) a carbonic anhydrase inhibitor (CAI), and a pharmaceutically acceptable carrier, and in the absence of an antimuscarinic agent.

Embodiments of this aspect of the invention may include one or more of the following optional features. In some embodiments, the NRI is a norepinephrine selective reuptake inhibitor (NSRI). In some embodiments, the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof. In some embodiments, the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof. In some embodiments, the NRI is reboxetine or a pharmaceutically acceptable salt thereof. In some embodiments, the NRI is atomoxetine or a pharmaceutically acceptable salt thereof. In some embodiments, the CAI is selected from the group consisting of acetazolamide, dichlorophenamide, dorzolamide, brinzolamide, methazolamide, zonisamide, ethoxzolamide, topiramate, sultiame, and any combination thereof, including pharmaceutically acceptable salts thereof. In some embodiments, the CAI is acetazolamide or a pharmaceutically acceptable salt thereof. In some embodiments, the NRI, such as atomoxetine or a pharmaceutically acceptable salt thereof, is present in an amount of from about 20 to about 200 mg. In some embodiments, the NRI, such as atomoxetine or a pharmaceutically acceptable salt thereof, is present in an amount of from about 25 to about 100 mg. In some embodiments, the CAI, such as acetazolamide, is present in an amount of from about 150 mg to about 750 mg. In some embodiments, the carbonic anhydrase inhibitor, such as acetazolamide, is present in an amount of about 500 mg. In some embodiments, the NRI and CAI are the sole active agents in the pharmaceutical composition. In some embodiments, the NRI and CAI are formulated in a single composition. In some embodiments, the single composition is an oral administration form. In some embodiments, the oral administration form is a syrup, pill, tablet, troche, capsule, or patch. In some embodiments, the pharmaceutical composition is for use in treating a subject having a condition associated with pharyngeal airway collapse. In some embodiments, the condition associated the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state. In some embodiments, the non-fully conscious state is sleep.

Another aspect of the present invention provides a pharmaceutical composition comprising (i) a hypnotic and (ii) a carbonic anhydrase inhibitor (CAI), and a pharmaceutically acceptable carrier, and in the absence of an antimuscarinic agent.

Embodiments of this aspect of the invention may include one or more of the following optional features. In some embodiments, the hypnotic is selected from the group consisting of temazepam, brotizolam, flurazepam, nitrazepam, and triazolam, and pharmaceutically acceptable salts thereof. In some embodiments, the hypnotic is selected from the group consisting of zolpidem, zopiclone, eszopiclone, gabapentin, trazodone, diphenhydramine, suvorexant, tasimelteon, ramelteon, agomelatine, doxepin, zaleplon, doxylamine, sodium oxybate, and tiagabine, and pharmaceutically acceptable salts thereof. In some embodiments, the hypnotic is trazodone. In some embodiments, the CAI is selected from the group consisting of acetazolamide, dichlorophenamide, dorzolamide, brinzolamide, methazolamide, zonisamide, ethoxzolamide, topiramate, sultiame, and any combination thereof, including pharmaceutically acceptable salts thereof. In some embodiments, the CAI is acetazolamide or a pharmaceutically acceptable salt thereof. In some embodiments, the trazodone is present in an amount of from about 12.5 to about 200 mg (or a dose equivalent of another hypnotic). In some embodiments, the trazodone is present in an amount of from about 12.5 to about 50 mg (or a dose equivalent of another hypnotic). In some embodiments, the trazodone is present in an amount of from about 50 to about 200 mg (or a dose equivalent of another hypnotic). In some embodiments, the trazodone present in an amount of from about 25 to about 100 mg (or a dose equivalent of another hypnotic). In some embodiments, the CAI, such as acetazolamide, is present in an amount of from about 150 mg to about 750 mg. In some embodiments, the carbonic anhydrase inhibitor, such as acetazolamide, is present in an amount of about 500 mg. In some embodiments, the hypnotic and CAI are the sole active agents in the pharmaceutical composition. In some embodiments, the hypnotic and CAI are formulated in a single composition. In some embodiments, the single composition is an oral administration form. In some embodiments, the oral administration form is a syrup, pill, tablet, troche, capsule, or patch. In some embodiments, the pharmaceutical composition is for use in treating a subject having a condition associated with pharyngeal airway collapse. In some embodiments, the condition associated the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state. In some embodiments, the non-fully conscious state is sleep.

Also provided herein is (i) a norepinephrine reuptake inhibitor (NRI) or a hypnotic and (ii) a carbonic anhydrase inhibitor (CAI), for use in treating a subject having a condition associated with pharyngeal airway collapse in the absence of an antimuscarinic therapy, and optionally wherein the agents (i) and (ii) are the sole active agents used to treat the condition.

Further provided herein is (i) a norepinephrine reuptake inhibitor (NRI) or a hypnotic and (ii) a carbonic anhydrase inhibitor (CAI), for use in treating sleep apnea in the absence of an antimuscarinic therapy, and optionally wherein the agents (i) and (ii) are the sole active agents used to treat sleep apnea.

Further provided herein is (i) a norepinephrine reuptake inhibitor (NRI) or a hypnotic and (ii) a carbonic anhydrase inhibitor (CAI), for use in treating snoring in the absence of an antimuscarinic therapy, and optionally wherein the agents (i) and (ii) are the sole active agents used to treat snoring.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and FIGURES, and from the claims.

### BRIEF DESCRIPTION OF THE DRAWINGS

The following FIGURES are provided by way of example and are not intended to limit the scope of the claimed invention.

FIG. 1 is a graphic illustration of an obstructive apnea. The top channel shows the electroencephalogram (EEG) pattern of sleep. The next channel represents airflow. The next three channels show ventilator effort by movements of the rib cage and abdomen and changes in esophageal pressure, all of which reflect a respiratory effort against an occluded upper airway. The last channel indicates oxyhemoglobin saturation.

### DETAILED DESCRIPTION

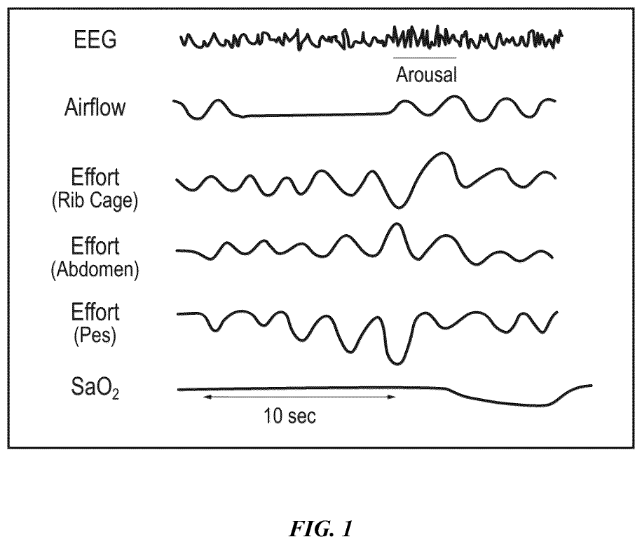

In humans, the pharyngeal airway region has no bone or cartilage support, and it is held open by muscles. When these muscles relax during sleep, the pharynx can collapse resulting in cessation of airflow. As shown in FIG. 1, ventilatory effort continues and increases in an attempt to overcome the obstruction, shown by an increase in esophageal pressure change. Rib cage and abdominal movements are in the opposite direction as a result of the diaphragm contracting against an occluded airway, forcing the abdominal wall to distend out and the chest wall to cave inward.

Increasing efforts to breathe lead to an arousal from sleep, visualisable on an EEG (FIG. 1), and result in opening of the airway and a resumption of normal breathing. The lack of airflow during the apnea also causes hypoxia, shown by a drop in oxyhemoglobin saturation (FIG. 1). Severity is generally measured using the apnea-hypopnea index (AHI), which is the combined average number of apneas (cessation of breathing for at least ten seconds) and hypopneas (reduced airflow and oxygen saturation) that occur per hour of sleep (Ruehland, W R. et al., The new AASM criteria for scoring hypopneas: Impact on the apnea hypopnea index. SLEEP 2009; 32(2):150-157).

FIG. 1 is a graphic illustration of an obstructive apnea. The top channel shows the electroencephalogram (EEG) pattern of sleep. The next channel represents airflow. The next three channels show ventilatory effort by movements of the rib cage and abdomen and changes in esophageal pressure, all of which reflect a respiratory effort against an occluded upper airway. The last channel indicates oxyhemoglobin saturation.

When a stringent definition of OSA is used (an AHI of >15 events per hour or AHI>5 events per hour with daytime sleepiness), the estimated prevalence is approximately 15 percent in males and 5 percent in females. An estimated 30 million individuals in the United States have OSA, of which approximately 6 million have been diagnosed. The prevalence of OSA in the United States appears to be increasing due to aging and increasing rates of obesity. OSA is associated with major comorbidities and economic costs, including: hypertension, diabetes, cardiovascular disease, motor vehicle accidents, workplace accidents, and fatigue/lost productivity. (Young, T. et al., WMJ 2009; 108:246; Peppard, P E. et al., Am J Epidemiol 2013; 177:1006.)

The present leading treatment is continuous positive airway pressure (CPAP). CPAP is effective in virtually all patients, and approximately 85% of diagnosed patients are prescribed CPAP, but compliance is low. Patients find CPAP uncomfortable and often intolerable; at least 30% of patients (up to 80%) are regularly non-adherent and thus untreated (Weaver, T E. Proc Am Thorac Soc. 2008 Feb. 15; 5(2): 173-178). Other treatment modalities with variable rates of success include oral appliances (10%) and surgery (5%), but neither is likely to be effective across the general population.

The search for medicines to activate pharyngeal muscles in sleeping humans has been discouraging; agents such as serotonin reuptake inhibitors, tricyclic antidepressants, and sedatives have all been tested in humans and shown to be ineffective at reducing OSA severity. See, e.g., Hudgel, D A. et al., Chest. 1991 August; 100(2):416-21; Brownell L G. et al., N Engl J Med 1982, 307:1037-1042; Sangal R B. et al., Sleep Med. 2008 July; 9(5):506-10. Epub 2007 Sep. 27; Marshall, N S. et al. Sleep 2008 June; 31(6):824-31; Eckert, D J. et al., Clin Sci (Lond). 2011 June; 120(12); 505-14; Taranto-Montemurro, L. et al., Sleep 2017 Feb. 1; 40(2):ZSW047.

In a recent study, a combination of atomoxetine and oxybutynin, referred to as “ato-oxy,” administered before bedtime has been shown to reduce OSA in patients with a wide range of severity. The ato-oxy combination, which was administered for one night, reduced the number of obstructive events, improved the overnight oxygen desaturation, and enhanced the genioglossus muscle activity in a group of unselected patients with OSA. The data collected in the proof-of-concept trial showed that it was possible to improve or abolish OSA using drugs with specific neurotransmitter profiles administered systemically. See Taranto-Montemurro, L. et al., The Combination of Atomoxetine and Oxybutynin Greatly Reduces Obstructive Sleep Apnea Severity. A Randomized, Placebo-controlled, Double-Blind Crossover Trial. Am J Respir Crit Care Med 2019 May 15; 199(10):1267-1276.

Acetazolamide is a respiratory stimulant used in healthy adults at high-altitude (where it augments and stabilizes respiration) and has additionally been administered safely to treat long-term for glaucoma and heart failure.

There remains a need for further therapies for treating conditions associated with pharyngeal airway collapse such as sleep apnea.

### Methods of Treatment

The methods described herein include methods for the treatment of disorders associated with pharyngeal airway muscle collapse during sleep. In some embodiments, the disorder is sleep apnea (e.g., obstructive sleep apnea (OSA)) or snoring (e.g., simple snoring). Generally, the methods include administering a therapeutically effective amount of (i) a norepinephrine reuptake inhibitor (e.g., atomoxetine or a pharmaceutically acceptable salt thereof) or a hypnotic (e.g., trazodone or a pharmaceutically acceptable salt thereof) and (ii) a carbonic anhydrase inhibitor (e.g., acetazolamide or a pharmaceutically acceptable salt thereof), to a subject who is in need of, or who has been determined to be in need of, such treatment.

Generally, the methods include administering an effective amount of (i) a norepinephrine reuptake inhibitor (NRI) or a hypnotic and (ii) a carbonic anhydrase inhibitor, in the absence of an antimuscarinic therapy, to a subject who is in need of, or who has been determined to be in need of, such treatment. In certain embodiments, the methods include administering an effective amount of (i) atomoxetine or a pharmaceutically acceptable salt thereof and (ii) acetazolamide or a pharmaceutically acceptable salt thereof, in the absence of an antimuscarinic therapy, to a subject who is in need of, or who has been determined to be in need of, such treatment. In certain embodiments, the methods include administering an effective amount of (i) trazodone or a pharmaceutically acceptable salt thereof and (ii) acetazolamide or a pharmaceutically acceptable salt thereof, in the absence of an antimuscarinic therapy, to a subject who is in need of, or who has been determined to be in need of, such treatment.

As used in this context, to “treat” means to ameliorate at least one symptom of the disorder associated with pharyngeal airway collapse. Often, pharyngeal airway collapse during sleep results in snoring and/or an interruption in breathing (apnea or hypopnea), arousal from sleep, and reduced oxygenation (hypoxemia); thus, a treatment can result in a reduction in snoring, apneas/hypopneas, sleep fragmentation, and hypoxemia. Administration of a therapeutically effective amount of a compound described herein for the treatment of a subject with OSA may result in decreased AHI. Measurement of OSA disease and symptoms may be, for example, by polysomnography (PSG).

In general, an “effective amount” of a compound refers to an amount sufficient to elicit the desired biological response, e.g., to treat a condition associated with pharyngeal airway collapse, e.g., to treat sleep apnea or snoring. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

An effective amount can be administered in one or more administrations, applications or dosages. The compositions can be administered from one or more times per day to one or more times per week; including once every other day. In some embodiments, the compositions are administered daily. In some embodiments, the compositions are administered daily before sleep time, e.g., immediately before sleep time or 15-60 minutes before sleep time. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, an “antimuscarinic therapy” refers to the administration of an antimuscarinic agent. Antimuscarinic agents include but are not limited to atropine, propantheline, bethanechol, solifenacin, darifenacin, tolterodine, fesoterodine, trospium, oxybutynin, anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl. Subjects receiving treatment according to the present disclosure in the absence of an antimuscarinic therapy do not receive administration of an antimuscarinic agent.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salts” include those derived from inorganic bases such as sodium, potassium, lithium, ammonium, calcium, magnesium, iron, zinc, copper, manganese, aluminum salts, and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

As used herein, the term “unit dosage form” is defined to refer to the form in which the compound is administered to a subject. Specifically, the unit dosage form can be, for example, a pill, capsule, or tablet. In some embodiments, the unit dosage form is a capsule.

As used herein, “solid dosage form” means a pharmaceutical dose(s) in solid form, e.g., tablets, capsules, granules, powders, sachets, reconstitutable powders, dry powder inhalers and chewables.

For the compounds disclosed herein, single stereochemical isomers, as well as enantiomers, diastereomers, cis/trans conformation isomers, and rotational isomers, and racemic and non-racemic mixtures thereof, are within the scope of the invention. Unless otherwise indicated, all tautomeric forms of the compounds disclosed herein are within the scope of the invention.

Atomoxetine is the generic name of the pharmaceutical substance with the chemical name (−)-N-Methyl-3-phenyl-3-(o-tolyloxy)-propylamine, and its pharmaceutical salts. Atomoxetine is the R(−)-isomer as determined by X-ray diffraction. In some embodiments, atomoxetine may be atomoxetine hydrochloride.

Acetazolamide is the generic name of the pharmaceutical substance with the chemical name N-(5-Sulfamoyl-1,3,4-thiadiazol-2-yl)acetamide, and its pharmaceutical salts. Acetazolamide is available as a generic medication as well as sold under the trade names Diamox, Dacarb, and others.

Trazodone is the generic name of the pharmaceutical substance with the chemical name 2-[3-[4-(3-chlorophenyl)piperazin-1-yl]propyl]-[1,2,4]triazolo[4,3-a]pyridin-3-one, and its pharmaceutical salts, including its hydrochloride salt. Trazodone is available as a generic medication as well as sold under the trade names Desyrel and Oleptro.

In some embodiments, the methods include administering a dose of from about 20 mg to about 200 mg of atomoxetine or a pharmaceutically acceptable salt thereof (or a dose equivalent of another NRI). In some embodiments, the dose of atomoxetine or a pharmaceutically acceptable salt thereof is from about 25 mg to about 100 mg. In some embodiments, the dose of atomoxetine or pharmaceutically acceptable salt thereof is from about 40 mg to about 80 mg. In some embodiments, the dose of atomoxetine or pharmaceutically acceptable salt thereof is from about 20 mg to about 50 mg. In some embodiments, the dose of atomoxetine or a pharmaceutically acceptable salt thereof is from about 50 mg to about 100 mg. In some embodiments, the dose of atomoxetine or pharmaceutically acceptable salt thereof is about 40 mg. In some embodiments, the dose of atomoxetine or pharmaceutically acceptable salt thereof is about 80 mg.

In some embodiments, the methods include administering a dose of from about 50 mg to about 1000 mg acetazolamide (or a dose equivalent thereof of another CAI), from about 100 mg to about 800 mg acetazolamide, from about 150 mg to about 750 mg acetazolamide, from about 250 mg to about 750 mg acetazolamide, from about 500 mg to about 750 mg acetazolamide, or from about 450 mg to about 650 mg acetazolamide.

In some embodiments, the methods include administering a dose of from about 12.5 to about 200 mg of trazodone or a pharmaceutically acceptable salt thereof (or a dose equivalent of another hypnotic). In some embodiments, the trazodone is administered at a dose of from about 12.5 to about 50 mg (or a dose equivalent of another hypnotic). In some embodiments, the trazodone is administered at a dose of from about 50 to about 200 mg (or a dose equivalent of another hypnotic). In some embodiments, the trazodone is administered at a dose of from about 25 to about 100 mg (or a dose equivalent of another hypnotic).

In some embodiments, the (i) norepinephrine reuptake inhibitor (NRI) and (ii) carbonic anhydrase inhibitor are the sole active agents administered in the methods described herein (e.g., without another active agent, such as an antimuscarinic agent and/or a complementary medicament or sleep aid). In some embodiments, (i) atomoxetine or a pharmaceutically acceptable salt thereof and (ii) acetazolamide or a pharmaceutically acceptable salt thereof, are the sole active agents administered in the methods described herein (e.g., without another active agent, such as an antimuscarinic agent and/or a complementary medicament or sleep aid).

In some embodiments, the (i) hypnotic and (ii) carbonic anhydrase inhibitor are the sole active agents administered in the methods described herein (e.g., without another active agent, such as an antimuscarinic agent and/or a complementary medicament or sleep aid). In some embodiments, (i) trazodone or a pharmaceutically acceptable salt thereof and (ii) acetazolamide or a pharmaceutically acceptable salt thereof are the sole active agents administered in the methods described herein (e.g., without another active agent, such as an antimuscarinic agent and/or a complementary medicament or sleep aid).

In some embodiments, the combination of (i) a norepinephrine reuptake inhibitor (NRI) and (ii) carbonic anhydrase inhibitor is administered with one or more additional active agents, which are not antimuscarinic agents, e.g., a hypnotic. In some embodiments, the combination of (i) atomoxetine or a pharmaceutically acceptable salt thereof and (ii) acetazolamide or a pharmaceutically acceptable salt thereof is administered with one or more additional active agents, which are not antimuscarinic agents, e.g., a hypnotic. Exemplary hypnotics include temazepam, brotizolam, flurazepam, nitrazepam, triazolam, zolpidem, zopiclone, eszopiclone, trazodone, zaleplon, benzodiazepines, gabapentin, tiagabine, and xyrem or pharmaceutically acceptable salts thereof.

### Pharmaceutical Compositions

Also provided herein are pharmaceutical compositions comprising (i) a norepinephrine reuptake inhibitor (NRI) or a hypnotic and (ii) a carbonic anhydrase inhibitor (CAI), as the sole active agents (e.g., without an antimuscarinic agent and/or a complementary medicament or sleep aid). The sole active ingredients can be in a single composition or in separate compositions. In certain embodiments, the pharmaceutical compositions include (i) atomoxetine or a pharmaceutically acceptable salt thereof and (ii) acetazolamide or a pharmaceutically acceptable salt thereof as sole active agents (e.g., without an antimuscarinic agent and/or a complementary medicament or sleep aid). In certain embodiments, the pharmaceutical compositions include (i) trazodone or a pharmaceutically acceptable salt thereof and (ii) acetazolamide or a pharmaceutically acceptable salt thereof as sole active agents (e.g., without an antimuscarinic agent and/or a complementary medicament or sleep aid).

Exemplary norepinephrine reuptake inhibitors (NRIs) include the selective NRIs, e.g., amedalin (UK-3540-1), atomoxetine (Strattera), CP-39,332, daledalin (UK-3557-15), edivoxetine (LY-2216684), esreboxetine, lortalamine (LM-1404), nisoxetine (LY-94,939), reboxetine (Edronax, Vestra), talopram (Lu 3-010), talsupram (Lu 5-005), tandamine (AY-23,946), viloxazine (Vivalan); and the non-selective NRIs, e.g., amitriptiline, amoxapine, bupropion, ciclazindol, desipramine, desvenlafaxine, dexmethilphenidate, diethylpropion, doxepin, duloxetine, imipramine, levomilnacipran, manifaxine (GW-320,659), maprotiline, methylphenidate, milnacipran, nefazodone, nortriptyline, phendimetrazine, phenmetrazine, protryptyline, radafaxine (GW-353,162), tapentadol (Nucynta), teniloxazine (Lucelan, Metatone) and venlafaxine; and pharmaceutically acceptable salts thereof.

In some embodiments, the NRI is atomoxetine or a pharmaceutically acceptable salt thereof. In some embodiments, the NRI is reboxetine or a pharmaceutically acceptable salt thereof.

Exemplary hypnotics include temazepam, brotizolam, flurazepam, nitrazepam, triazolam, zolpidem, zopiclone, eszopiclone, gabapentin, trazodone, diphenhydramine, suvorexant, tasimelteon, ramelteon, agomelatine, doxepin, zaleplon, doxylamine, sodium oxybate, and tiagabine; and pharmaceutically acceptable salts thereof.

Exemplary carbonic anhydrase inhibitor (CAI) include acetazolamide, dichlorophenamide, dorzolamide, brinzolamide, methazolamide, zonisamide, ethoxzolamide, topiramate, sultiame, and any combinations thereof, or a pharmaceutically acceptable salt thereof.

In some embodiments, the carbonic anhydrase inhibitor is acetazolamide or a pharmaceutically acceptable salt thereof.

Pharmaceutical compositions typically include a pharmaceutically acceptable carrier. As used herein the language “pharmaceutically acceptable carrier” includes saline, solvents, dispersion media, diluents, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include systemic oral, transdermal administration, and parenteral administration.

Methods of formulating suitable pharmaceutical compositions using pharmaceutically acceptable carriers are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. In some embodiments, a composition according to the present invention may be a unit dosage form. In some embodiments, a composition according to the present invention may be a solid dosage form, e.g., a tablet or capsule.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of the compounds as described herein can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 April; 8(2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

The pharmaceutical compositions can be included in a container, pack, or dispenser together with instructions for administration or use in a method described herein.

In some embodiments, the (i) norepinephrine reuptake inhibitor (NRI) and (ii) carbonic anhydrase inhibitor, are the sole active agents present in the pharmaceutical composition. In some embodiments, (i) atomoxetine or a pharmaceutically acceptable salt thereof and (ii) acetazolamide or a pharmaceutically acceptable salt thereof, are the sole active agents present in the pharmaceutical composition. In some embodiments, the pharmaceutical composition does not contain another active agent (e.g., an antimuscarinic agent and/or a complementary medicament or sleep aid).

In some embodiments, the pharmaceutical composition is for use in treating a condition associated with pharyngeal airway muscle collapse during sleep. In some embodiments, the condition associated with pharyngeal airway muscle collapse is sleep apnea (e.g., obstructive sleep apnea (OSA)) or snoring (e.g., simple snoring).

### Combinations

Also provided herein is (i) a norepinephrine reuptake inhibitor (NRI) or a hypnotic and (ii) a carbonic anhydrase inhibitor (CAI), for use in treating a subject having a condition associated with pharyngeal airway collapse in the absence of an antimuscarinic therapy, and optionally wherein the agents (i) and (ii) are the sole active agents. In some embodiments, the condition associated with pharyngeal airway muscle collapse is sleep apnea (e.g., obstructive sleep apnea (OSA)) or snoring (e.g., simple snoring). In some embodiments, the combination does not contain another active agent (e.g., an antimuscarinic agent and/or a complementary medicament or sleep aid). In some embodiments, the combination contains an NRI, a CAI, and a hypnotic.

Further provided herein is (i) a norepinephrine reuptake inhibitor (NRI) or a hypnotic and (ii) a carbonic anhydrase inhibitor (CAI), for use in treating sleep apnea in the absence of an antimuscarinic therapy, and optionally wherein the agents (i) and (ii) are the sole active agents. In some embodiments, the combination does not contain another active agent (e.g., an antimuscarinic agent and/or a complementary medicament or sleep aid). In some embodiments, the combination contains an NRI, a CAI, and a hypnotic.

Also provided herein is (i) a norepinephrine reuptake inhibitor (NRI) or a hypnotic and (ii) a carbonic anhydrase inhibitor (CAI), for use in treating snoring in the absence of an antimuscarinic therapy, and optionally wherein the agents (i) and (ii) are the sole active agents. In some embodiments, the combination does not contain another active agent (e.g., an antimuscarinic agent and/or a complementary medicament or sleep aid). In some embodiments, the combination contains an NRI, a CAI, and a hypnotic.

### EXAMPLES

The invention is further described in the following examples, which do not limit the scope of the invention described in the claims.

### Example 1. Atomoxetine+Acetazolamide Crossover Study

A placebo-controlled, double-blinded, randomized, crossover trial in OSA human patients can be performed. Participants would receive treatment (once daily atomoxetine plus acetazolamide or placebo) in randomized order 30 minutes before sleep. The treatment would be evaluated for its ability to reduce the apnea hypopnea index and improve OSA severity. Additional benefits evaluated would be increased genioglossus muscle responsiveness to an increase in ventilatory drive, improved upper airway muscle activity, improved ventilation, increased oxygen levels (SaO₂), increased total sleep time, and improved sleep efficiency.

### Example 2. Reboxetine+Acetazolamide Crossover Study

A placebo-controlled, double-blinded, randomized, crossover trial in OSA human patients can be performed. Participants would receive treatment (once daily reboxetine plus acetazolamide or placebo) in randomized order 30 minutes before sleep. The treatment would be evaluated for its ability to reduce the apnea hypopnea index and improve OSA severity. Additional benefits evaluated would be increased genioglossus muscle responsiveness to an increase in ventilatory drive, improved upper airway muscle activity, improved ventilation, increased oxygen levels (SaO₂), increased total sleep time, and improved sleep efficiency.

### Example 3. Trazodone+Acetazolamide Crossover Study

A placebo-controlled, double-blinded, randomized, crossover trial in OSA human patients can be performed. Participants would receive treatment (once daily trazodone plus acetazolamide or placebo) in randomized order 30 minutes before sleep. The treatment would be evaluated for its ability to reduce the apnea hypopnea index and improve OSA severity. Additional benefits evaluated would be increased genioglossus muscle responsiveness to an increase in ventilatory drive, improved upper airway muscle activity, improved ventilation, increased oxygen levels (SaO₂), increased total sleep time, and improved sleep efficiency.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a condition associated with pharyngeal airway collapse, the method comprising administering to a subject in need thereof an effective amount of (i) a norepinephrine reuptake inhibitor (NRI) and (ii) a carbonic anhydrase inhibitor (CAI) in the absence of an antimuscarinic therapy.

2. The method of claim 1, wherein the NRI is a norepinephrine selective reuptake inhibitor (NSRI).

3. The method of claim 2, wherein the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof.

4. The method of claim 1, wherein the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof.

5. The method of claim 1, wherein the NRI is reboxetine or a pharmaceutically acceptable salt thereof.

6. The method of claim 1, wherein the NRI is atomoxetine or a pharmaceutically acceptable salt thereof.

7. The method of any one of claims 1-6, wherein the CAI is selected from the group consisting of acetazolamide, dichlorophenamide, dorzolamide, brinzolamide, methazolamide, zonisamide, ethoxzolamide, topiramate, sultiame, and any combination thereof, or a pharmaceutically acceptable salt thereof.

8. The method of claim 7, wherein the CAI is acetazolamide or a pharmaceutically acceptable salt thereof.

9. The method of any one of claims 1-8, wherein the NRI, such as atomoxetine or a pharmaceutically acceptable salt thereof, is administered at a dose of from about 20 to about 200 mg.

10. The method of claim 9, wherein the NRI, such as atomoxetine or a pharmaceutically acceptable salt thereof, is administered at a dose of from about 25 to about 100 mg.

11. The method of any one of claims 1-10, wherein the CAI, such as acetazolamide, is administered at a dosage of from about 150 mg to about 750 mg.

12. The method of claim 11, wherein the carbonic anhydrase inhibitor, such as acetazolamide, is administered at a dosage of about 500 mg.

13. The method of any one of claims 1-12, wherein the NRI and CAI are the sole active agents.

14. The method of any one of claims 1-13, wherein the NRI and CAI are administered in a single composition.

15. The method of claim 14, wherein the single composition is an oral administration form.

16. The method of claim 15, wherein the oral administration form is a syrup, pill, tablet, troche, capsule, or patch.

17. The method of any one of claims 1-16, wherein the condition associated with pharyngeal airway collapse is sleep apnea.

18. The method of claim 17, wherein the condition associated with pharyngeal airway collapse is obstructive sleep apnea (OSA).

19. The method of any one of claims 1-16, wherein the condition associated with pharyngeal airway collapse is snoring.

20. The method of claim 19, wherein the condition associated with pharyngeal airway collapse is simple snoring.

21. The method of any one of claims 1-20, wherein the subject is in a non-fully conscious state.

22. The method of claim 21, wherein the non-fully conscious state is sleep.

23. A pharmaceutical composition comprising (i) a norepinephrine reuptake inhibitor (NRI) and (ii) a carbonic anhydrase inhibitor (CAI), and a pharmaceutically acceptable carrier and in the absence of an antimuscarinic agent.

24. The pharmaceutical composition of claim 23, wherein the NRI is a norepinephrine selective reuptake inhibitor (NSRI).

25. The pharmaceutical composition of claim 24, wherein the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof.

26. The pharmaceutical composition of claim 23, wherein the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof.

27. The pharmaceutical composition of claim 23, wherein the NRI is reboxetine or a pharmaceutically acceptable salt thereof.

28. The pharmaceutical composition of claim 23, wherein the NRI is atomoxetine or a pharmaceutically acceptable salt thereof.

29. The pharmaceutical composition of any one of claims 23-28, wherein the CAI is selected from the group consisting of acetazolamide, dichlorophenamide, dorzolamide, brinzolamide, methazolamide, zonisamide, ethoxzolamide, topiramate, sultiame, or a pharmaceutically acceptable salt thereof.

30. The pharmaceutical composition of claim 29, wherein the CAI is acetazolamide or a pharmaceutically acceptable salt thereof.

31. The pharmaceutical composition of any one of claims 23-30, wherein the NRI, such as atomoxetine or pharmaceutically acceptable salt thereof, is present in an amount of from about 20 to about 200 mg.

32. The pharmaceutical composition of claim 31 wherein the NRI, such as atomoxetine or pharmaceutically acceptable salt thereof, is present in an amount of from about 25 to about 100 mg.

33. The pharmaceutical composition of any one of claims 23-32, wherein the CAI, such as acetazolamide, is present in an amount of from about 150 mg to about 750 mg.

34. The pharmaceutical composition of claim 33, wherein the CAI, such as acetazolamide, is present in an amount of about 500 mg.

35. The pharmaceutical composition of any one of claims 23-34, wherein the NRI and CAI are the sole active agents.

36. The pharmaceutical composition of any one of claims 23-35, wherein the NRI and CAI are in a single composition.

37. The pharmaceutical composition of claim 36, wherein the single composition is an oral administration form.

38. The pharmaceutical composition of claim 37, wherein the oral administration form is a syrup, pill, tablet, troche, capsule, or patch.

39. The pharmaceutical composition of any one of claims 23-38, for use in treating a subject having a condition associated with pharyngeal airway collapse.

40. The pharmaceutical composition of claim 39, wherein the condition associated with pharyngeal airway collapse is sleep apnea.

41. The pharmaceutical composition of claim 40, wherein the condition associated with pharyngeal airway collapse is obstructive sleep apnea (OSA).

42. The pharmaceutical composition of claim 39, wherein the condition associated with pharyngeal airway collapse is snoring.

43. The pharmaceutical composition of claim 42, wherein the condition associated with pharyngeal airway collapse is simple snoring.

44. The pharmaceutical composition of any one of claims 39-43, wherein the subject is in a non-fully conscious state.

45. The pharmaceutical composition of claim 44, wherein the non-fully conscious state is sleep.

46. A norepinephrine reuptake inhibitor (NRI) and a carbonic anhydrase inhibitor (CAI) for use in treating a subject having a condition associated with pharyngeal airway collapse in the absence of an antimuscarinic therapy, and optionally wherein the NRI and CAI are the sole active agents.

47. A norepinephrine reuptake inhibitor (NRI) and a carbonic anhydrase inhibitor (CAI) for use in treating sleep apnea in the absence of an antimuscarinic therapy, and optionally wherein the NRI and CAI are the sole active agents.

48. A norepinephrine reuptake inhibitor (NRI) and a carbonic anhydrase inhibitor (CAI) for use in treating snoring in the absence of an antimuscarinic therapy, and optionally wherein the NRI and CAI are the sole active agents.

49. A method of treating a subject having a condition associated with pharyngeal airway collapse, the method comprising administering to a subject in need thereof an effective amount of (i) a hypnotic and (ii) a carbonic anhydrase inhibitor (CAI) in the absence of an antimuscarinic therapy.

50. The method of claim 49, wherein the hypnotic is selected from the group consisting of zolpidem, zopiclone, eszopiclone, gabapentin, trazodone, diphenhydramine, suvorexant, tasimelteon, ramelteon, agomelatine, doxepin, zaleplon, doxylamine, sodium oxybate, and tiagabine, or a pharmaceutically acceptable salt thereof.

51. The method of claim 50, wherein the hypnotic is trazodone or a pharmaceutically acceptable salt thereof.

52. The method of any one of claims 49-51, wherein the CAI is selected from the group consisting of acetazolamide, dichlorophenamide, dorzolamide, brinzolamide, methazolamide, zonisamide, ethoxzolamide, topiramate, sultiame, and any combination thereof, or a pharmaceutically acceptable salt thereof.

53. The method of claim 52, wherein the CAI is acetazolamide or a pharmaceutically acceptable salt thereof.

54. The method of any one of claim 51, wherein trazodone is administered at a dose of from about 12.5 to about 200 mg.

55. The method of claim 53 or 54, wherein acetazolamide is administered at a dosage of from about 150 mg to about 750 mg.

56. The method of any one of claims 49-55, wherein the hypnotic and CAI are the sole active agents.

57. The method of any one of claims 49-56, wherein the hypnotic and CAI are administered in a single composition.

58. The method of claim 57, wherein the single composition is an oral administration form.

59. The method of claim 58, wherein the oral administration form is a syrup, pill, tablet, troche, capsule, or patch.

60. The method of any one of claims 49-59, wherein the condition associated with pharyngeal airway collapse is sleep apnea.

61. The method of claim 60, wherein the condition associated with pharyngeal airway collapse is obstructive sleep apnea (OSA).

62. The method of any one of claims 49-59, wherein the condition associated with pharyngeal airway collapse is snoring.

63. The method of claim 62, wherein the condition associated with pharyngeal airway collapse is simple snoring.

64. The method of any one of claims 49-63, wherein the subject is in a non-fully conscious state.

65. The method of claim 64, wherein the non-fully conscious state is sleep.

66. A pharmaceutical composition comprising (i) a hypnotic and (ii) a carbonic anhydrase inhibitor (CAI), and a pharmaceutically acceptable carrier and in the absence of an antimuscarinic agent.

67. The pharmaceutical composition of claim 66, wherein the hypnotic is selected from the group consisting of zolpidem, zopiclone, eszopiclone, gabapentin, trazodone, diphenhydramine, suvorexant, tasimelteon, ramelteon, agomelatine, doxepin, zaleplon, doxylamine, sodium oxybate, and tiagabine, or a pharmaceutically acceptable salt thereof.

68. The pharmaceutical composition of claim 67, wherein the hypnotic is trazodone or a pharmaceutically acceptable salt thereof.

69. The pharmaceutical composition of any one of claims 66-68, wherein the CAI is selected from the group consisting of acetazolamide, dichlorophenamide, dorzolamide, brinzolamide, methazolamide, zonisamide, ethoxzolamide, topiramate, sultiame, or a pharmaceutically acceptable salt thereof.

70. The pharmaceutical composition of claim 69, wherein the CAI is acetazolamide or a pharmaceutically acceptable salt thereof.

71. The pharmaceutical composition of claim 68, wherein the trazodone or pharmaceutically acceptable salt thereof is present in an amount of 12.5 mg to 200 mg.

72. The pharmaceutical composition of claim 70 or 71, wherein the acetazolamide is present in an amount of from about 150 mg to about 750 mg.

73. The pharmaceutical composition of any one of claims 66-72, wherein the hypnotic and CAI are the sole active agents.

74. The pharmaceutical composition of any one of claims 66-73, wherein the hypnotic and CAI are in a single composition.

75. The pharmaceutical composition of claim 74, wherein the single composition is an oral administration form.

76. The pharmaceutical composition of claim 75, wherein the oral administration form is a syrup, pill, tablet, troche, capsule, or patch.

77. The pharmaceutical composition of any one of claims 66-76, for use in treating a subject having a condition associated with pharyngeal airway collapse.

78. The pharmaceutical composition of claim 77, wherein the condition associated with pharyngeal airway collapse is sleep apnea.

79. The pharmaceutical composition of claim 78, wherein the condition associated with pharyngeal airway collapse is obstructive sleep apnea (OSA).

80. The pharmaceutical composition of claim 77, wherein the condition associated with pharyngeal airway collapse is snoring.

81. The pharmaceutical composition of claim 80, wherein the condition associated with pharyngeal airway collapse is simple snoring.

82. The pharmaceutical composition of any one of claims 77-81, wherein the subject is in a non-fully conscious state.

83. The pharmaceutical composition of claim 82, wherein the non-fully conscious state is sleep.

84. A hypnotic and a carbonic anhydrase inhibitor (CAI) for use in treating a subject having a condition associated with pharyngeal airway collapse in the absence of an antimuscarinic therapy, and optionally wherein the hypnotic and CAI are the sole active agents.

85. A hypnotic and a carbonic anhydrase inhibitor (CAI) for use in treating sleep apnea in the absence of an antimuscarinic therapy, and optionally wherein the hypnotic and CAI are the sole active agents.

86. A hypnotic and a carbonic anhydrase inhibitor (CAI) for use in treating snoring in the absence of an antimuscarinic therapy, and optionally wherein the hypnotic and CAI are the sole active agents.
